(19) United States
(12) Patent Application Publication
(10) Pub. No.: US 2026/0199560 A1
(43) Pub. Date: Jul. 16, 2026

(54) Methods For Modulating Lymphatic Pathway of Central Nervous System

(71) Applicant: Fibralign Corporation, Union City, CA (US)

(72) Inventors: Michael Paukshto, Foster City, CA (US); Dimitrios Dionyssiou, Thessaloniki (GR)

(21) Appl. No.: 19/136,265

(22) PCT Filed: Dec. 7, 2023
(86) PCT No.: PCT/US2023/082984

§ 371 Date: Jun. 5, 2025

Related U.S. Application Data
(60) Provisional application No. 63/386,406, filed on Dec. 7, 2022.

(51) Int. Cl. A61L 27/58; A61L 27/24; A61L 27/38; A61L 27/54

(52) U.S. Cl. CPC A61L 27/58; A61L 27/24; A61L 27/3808; A61L 27/3834; A61L 27/54; A61L 2300/258; A61L 2300/414; A61L 2400/06; A61L 2400/12

(57) ABSTRACT

In some embodiments herein, implantable scaffolds, their compositions, and methods for modulating lymphatic pathway in the central nervous system and in the vicinity of the central nervous system are presented. Modulating lymphatic pathway, increasing the flow and turnover of fluid in the central nervous system, and, in particular, inducing formation of new lymphatic vessels, in accordance with some embodiments, are used to treat, prevent, or ameliorate symptoms of neurodegenerative diseases.

### CROSS-REFERENCE TO RELATED APPLICATIONS

This application claims the benefit of and priority to U.S. Provisional Application No. 63/386,406, filed on Dec. 7, 2022, the content of which is incorporated herein by reference in its entirety.

### FIELD OF THE INVENTION

The invention relates generally to procedures and devices that facilitate the increase in turnover and flow of cerebrospinal fluid in the central nervous system for the purpose of treating disease, such as Alzheimer's disease, Parkinson's disease, and other neurological and psychiatric disorders. In particular, the invention relates to a method of implanting a biodegradable device that induces formation of complementary lymphatic pathways or/and increases capacity of the lymphatic system in the head and/or neck.

### BACKGROUND

Alzheimer's disease (AD) is the most frequent neurodegenerative disorder and most common cause of dementia in the elderly. Due to the increasingly aging population, the prevalence of AD dementia patients is expected to grow exponentially in the upcoming years.

Dementia due to AD is characterized by the accumulation of pathological Amyloid-Beta (Aβ) and Tau proteins, with potential neurodegeneration as a result. This agglomeration is increased by impaired clearance of waste from the brain. Therefore, an improvement of the clearance system is crucial for AD treatment [references 24,25]. This invention provides a therapeutic approach to treating Alzheimer's disease that emerged from the understanding that plaques and tangles causative but mere consequences of an underlying mechanism in which the clearance of toxic factors from the brain has been disrupted by genetic and/or environmental factors.

Several neurodegenerative diseases are characterized by intracellular or extracellular accumulation of protein aggregates and various metabolic debris [reference 1]. In recent years, there has been a sudden shift in our understanding of how the lymphatic system may be involved in this process in AD and other neurological disorders [references 2, 3]. After years of focusing almost exclusively on the blood-brain barrier, researchers found that brain lymphatic drainage is important for removing beta-amyloid, one of the neuropathological hallmarks of AD [references 4, 5].

Accordingly, significant developments and treatments are greatly needed.

### SUMMARY OF THE INVENTION

Head and neck lymphatic system consists of lymph nodes and lymphatic vessels. A part of this lymphatic system receives CSF (cerebrospinal fluid) and/or ISF (interstitial fluid) from CNS (central nervous system). We call this part—CSF drainage (lymphatic) system. It is important for parenchymal waste clearance, brain homeostasis, and the regulation of immune and inflammatory processes within the brain. There are a few lymph nodes in the CSF drainage system that are first to receive the CSF and/or ISF. These are the principal lymph nodes, dcLNs—deep cervical lymph nodes, involved in the immune response to CNS-derived antigens. Same CNS-derived antigens may cause a cytotoxic immune response in some other part of lymphatic system, for example, in some scLNs (superficial cervical lymph nodes) [references 26]. Therefore, it is important to detect and use the principal lymph nodes as a primary recipient of CSF. The current invention described herein aims at reconstruction and repair of the drainage lymphatic system in head and neck to improve parenchymal waste clearance, brain homeostasis, and the regulation of immune and inflammatory processes within the brain.

The proposed inventive therapeutic interventions described herein may reduce risks and complications associated with AD as well as other neurological and psychiatric conditions, e.g., Parkinson's disease, frontal-temporal dementia, mild cognitive impairment, idiopathic dementia, vascular dementia, amyotrophic lateral sclerosis, and concussive brain injury.

In some embodiments, the invention provides a method to treat neurological disorders and psychiatric conditions by increasing turnover and/or flow of cerebrospinal fluid in the central nervous system. The treatment is based on a formation of complementary lymphatic pathways via directional lymphatic regeneration and/or increasing a capacity of the lymphatic system of the head and neck. In some embodiments herein, implantable scaffolds, their compositions, and methods for modulating lymphatic pathway in the central nervous system and in the vicinity of the central nervous system are presented. Modulating lymphatic pathway and, in particular, inducing formation of new lymphatic vessels, in accordance with some embodiments, are used to treat, prevent, or ameliorate symptoms of neurodegenerative diseases, including Alzheimer's disease, Parkinson's disease, frontal-temporal dementia, mild cognitive impairment, idiopathic dementia, vascular dementia, amyotrophic lateral sclerosis, concussive brain injury, multiple sclerosis, as well as other neurological and psychiatric disorders.

For example, in one embodiment a method of increasing flow of fluid in the central nervous system of a subject in need thereof is provided, the method comprising: implanting a biocompatible scaffold into the subject head or neck wherein the scaffold induces a directional lymphatic regeneration and formation of a complementary lymphatic pathway, thereby resulting in an additional flow of cerebrospinal and/or extracellular fluid from the central nervous system into the circulatory system of the subject. In some embodiments, the additional flow is in the range of approximately 1%-10% increase flow of cerebrospinal and/or extracellular fluid from the central nervous system into the circulatory system of the subject.

In another example, a method of increasing turnover of cerebrospinal fluid of a subject in need thereof is provided, the method comprising: implanting a biocompatible scaffold into the subject head or neck wherein the scaffold induces a directional lymphangiogenesis, thereby resulting in an increase 1%-10% increase in the turnover of the subject cerebrospinal fluid. In some embodiments, the increase is more than 3% increase in the turnover of the subject cerebrospinal fluid. The clinical data demonstrate that ventricular tracer clearance in elderly individuals on average is more than 3% slower than in the healthy controls [reference 27] indicating a proportional decrease in the CSF turnover.

In some embodiments, the scaffold is configured to support flow of cerebrospinal and/or extracellular fluid of the subject and induces a formation of a complementary lymphatic pathway from the dura mater to the head and neck circulatory system of the subject, thereby increasing fluid flow in the central nervous system of the subject. The scaffold may be comprised of a thread-like biodegradable scaffold or a set of the thread-like biodegradable scaffolds. In some embodiments, the scaffold is an injectable scaffold. In some embodiments, the thread-like scaffold comprises a bundle of threads, or multi-filaments, or fibers, or fibrils made from biodegradable materials such that the bundle supports a capillary flow of cerebrospinal and extracellular fluid along the bundle, in the bundle direction. Additionally, the thread-like scaffold may be comprised of a multi-lumen or multi-channel structure such that the thread-like scaffold enabling a flow of cerebrospinal and extracellular fluid in the thread-like scaffold direction.

### BRIEF DESCRIPTION OF THE FIGURES

Embodiments of the present invention are illustrated by way of example and not limitation in the figures of the accompanying drawings, in which like references indicate similar elements and in which:

FIG. 1 is a schematic diagram of the routes for drainage of CSF and/or ISF with implanted thread-like scaffolds (indicated as BB) according to some embodiments. Option 1—the thread-like scaffold implanted under the skin of the subject head such that the first end of the thread-like scaffold is directed to the functional lymphatics of the “head and neck lymphatic system” and the second end of the thread-like scaffold goes through the skull into the dura mater in the proximity of the meningeal lymphatics. Option 2—the thread-like scaffold implanted such that the first end of the thread-like scaffold is inserted into the proximity of the ventral/inferior border of the brain surface near the functional lymphatics and the second end of the thread-like scaffold goes down into a functional cervical lymph node.

FIG. 2 is a schematic diagram showing the routes for drainage of CSF and/or ISF with implanted lymph node (VLNT procedure or a propeller lymphatic tissue flap [reference 16]) and thread-like scaffolds (indicated as BB) according to some embodiments. The scaffolds implanted to augment the transferred lymph node and improve its integration into lymphatic system. In the presented example the scaffold is bridging the transferred lymph node with a deep cervical lymph node which has a viable afferent and unhealthy efferent lymphatic channels.

FIG. 3 is a schematic diagram of the routes for drainage of CSF and/or ISF with implanted lymph node (VLNT procedure or a propeller lymphatic tissue flap) and thread-like scaffolds (indicated as BB) according to some embodiments. The scaffolds implanted to augment the transferred lymph node and improve its integration into lymphatic system. The scaffolds are implanted to connect the lymphatic at the vicinity of the ventral/inferior border of the brain surface with the implanted lymph node and the implanted lymph node with a subject healthy lymph node in the neck.

FIG. 4 is a schematic diagram illustrating the routes for drainage of CSF and/or ISF with implanted thread-like scaffolds (indicated as BB) and LVA in the neck region connecting a healthy lymphatic vessel going from the ventral/inferior border of the brain with a vein according to some embodiments. The scaffold is implanted to enhance the flow of lymph fluid from meningeal lymphatic to the LVA area.

FIG. 5 is a schematic diagram of the routes for drainage of CSF and/or ISF with implanted thread-like scaffolds (indicated as BB) and LVA in the neck region connecting a healthy lymphatic vessel going from the ventral/inferior border of the brain with a vein according to some embodiments. The scaffold is implanted to connect a deep cervical lymph node with degraded efferent lymphatic vessel to the LVA area enhancing CNS antigen surveillance.

FIG. 6 is a schematic diagram showing the routes for drainage of CSF and ISF with implanted thread-like scaffolds (indicated as BB) and LVA in the neck region. The scaffolds are implanted to connect a deep cervical lymph node with degraded efferent lymphatic vessel to the LVA area enhancing CNS antigen surveillance and to enhance the flow of lymph fluid from meningeal lymphatic to the LVA area.

FIG. 7a-7e illustrate thread-like scaffold BioBridge™ in various embodiments of the present invention: a) the macroscopic view; b) the aligned nanofibrillar collagen structure of the scaffold; c) the surface of the scaffold; d) the cross-section of the scaffold with multilumen structure; e) the surface of the lumens with indicated aligned nanofibrillar collagen structure.

### DETAILED DESCRIPTION

Herein we define several terms used throughout the description, including: brain clearance system which includes meningeal lymphatic vessels and the glymphatic system; directional lymphatic regeneration which means a formation of lymphatic vessel along a specific direction, for example, along the gradient of VEGF-C growth factor; circulatory system which includes the blood circulatory system and lymphatic system; head and neck circulatory system; head and neck lymphatic system; nucleic acid, for example, modified mRNA like HGF-mRNA or VEGFC-mRNA; VLNT—Vascularized Lymph Node Transfer which is a transplantation of autologous functional lymph node with microanastomosis to vasculature in the recipient bed to maintain their blood supply; LVA—Lymphatico Venous Anastomosis which is a reconstructive surgical procedure to redirect lymphatic fluid into the venous system; CSF turnover which is defined as CSF production divided by its distribution volume; thread-like scaffold is the scaffold having a shape of thread or suture; injectable scaffold is the scaffold that can be formed from a solution or a gel after injection by a needle, for example, suspension of microspheres mixed with human VEGFC/D, or fibroblast growth factor, or hepatocyte growth factor, or platelet-derived growth factor, or insulin-like growth factor.

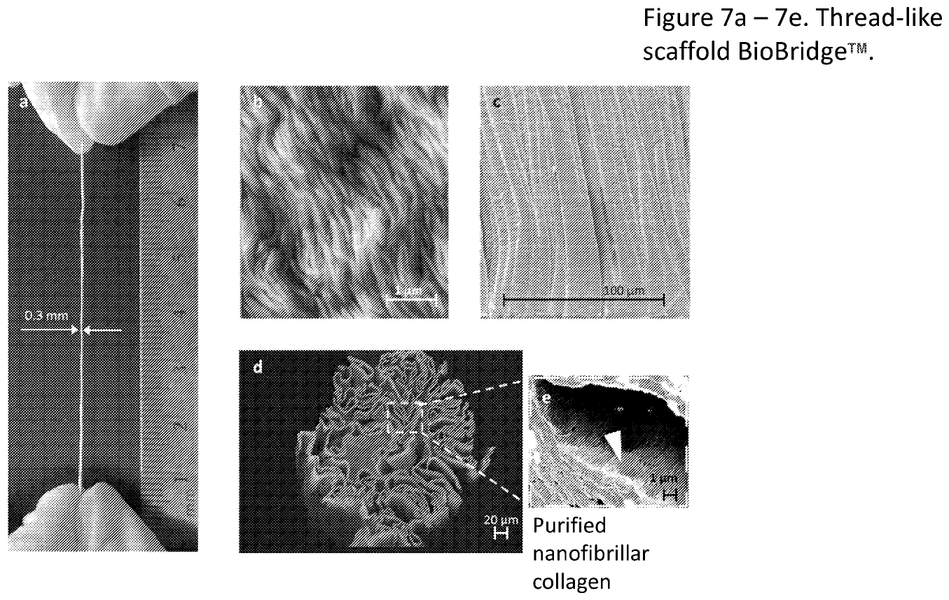

Disclosed herein, in certain embodiments, is a biocompatible scaffold which is made from anyone or more of: collagen, fibronectin, fibrin, laminin, elastin, hyaluronic acid, chitosan, silk, peptides, block copolymers, lactide and glycolide polymers, caprolactone polymers, hydroxybutyric acids, polyanhydrides and polyesters, polyphosphates, porous silicon, polyphosphoesters, poly(ethylene glycol) (PEG) and poly(ethylene oxide) (PEO) including PEG and PEO with different end functionalities, as well as bifunctional cross-linkers and crosslinking agents, or the combinations of the above materials. In another embodiment, the biodegradable and biocompatible scaffold is comprised of an aligned nanofibrillar biopolymer, for example, biopolymer composed of aligned nanofibrillar collagen type I or type III. One example of biocompatible biodegradable thread-like scaffold is the aligned nanofibrillar collagen scaffold (BioBridge™) presented in FIG. 7. This scaffold is used for lymphedema treatment and prevention. It demonstrated the ability to induce directional lymphatic regeneration and create a complementary lymphatic pathway in animal models and in human [references 17,18,20-23].

Disclosed herein, some embodiments include a method of increasing flow and/or turnover of fluid in the central nervous system of a subject. In one embodiment, the method comprises biocompatible scaffold implanted into the head of a subject wherein the scaffold supports a flow of cerebrospinal and/or extracellular fluid into the lymphatic system of the subject and induces a formation of a complementary lymphatic pathway from the head to at least one lymph node of the head or neck of the subject, thereby increasing fluid flow in the central nervous system of the subject. In other embodiment, the method comprises the scaffolds supporting a flow of cerebrospinal and extracellular fluid into the lymphatic system of the subject and induces a formation of a complementary lymphatic pathway from the meningeal lymphatics to at least one lymph nodes of the head or neck of the subject, thereby increasing fluid flow and/or turnover in the central nervous system of the subject.

In one embodiment, the scaffold is a thread-like biodegradable scaffold or a set of thread-like biodegradable scaffolds. In another embodiment, the scaffold is an injectable scaffold. In one embodiment, the thread-like scaffold comprises a bundle of threads, or multi-filaments, or fibers, or fibrils made from biodegradable materials such that the bundle supports a capillary flow of cerebrospinal and/or extracellular fluid in the bundle direction. In another embodiment, the thread-like scaffold has a multi-lumen or multi-channel structure such that the thread-like scaffold enabling a flow of cerebrospinal and/or extracellular fluid in the thread-like scaffold direction.

In several embodiments, the thread-like scaffold and injectable scaffold comprise biodegradable materials selected from the group consisting of collagen, fibronectin, fibrin, laminin, elastin, hyaluronic acid, chitosan, silk, peptides, block copolymers, lactide and glycolide polymers, caprolactone polymers, hydroxybutyric acids, polyanhydrides and polyesters, polyphosphates, porous silicon, polyphosphoesters, poly(ethylene glycol) (PEG) and poly(ethylene oxide) (PEO) including PEG and PEO with different end functionalities, as well as bifunctional cross-linkers and crosslinking agents, or the combinations of the above materials.

In several embodiments, the injectable and thread-like scaffolds comprising a growth factor or cells or immunotherapeutic drug, which may include human VEGFC/D, or fibroblast growth factor, or hepatocyte growth factor, or platelet-derived growth factor, or insulin-like growth factor, or other lymphangiogenesis promoting growth factors.

In several embodiments, the injectable and thread-like scaffolds comprising nucleic acid or cells promoting lymphangiogenesis. In particular, nucleic acid is a modified messenger RNA promoting lymphangiogenesis, for example, VEGFC/D-mRNA or FGF-mRNA or HGF-mRNA or PDGF-mRNA or IGF-mRNA or their combinations. The cells promoting lymphangiogenesis are, for example, autologous or allogeneic endothelial or endothelial progenitor cells.

In various embodiments, the device and procedure can be used to treat neurological and psychiatric diseases associated with disruptions of cerebrospinal fluid flow and drainage, including Alzheimer's disease, Parkinson's disease, frontal-temporal dementia, mild cognitive impairment, idiopathic dementia, vascular dementia, concussive brain injury, stroke, post-traumatic brain injury, multiple sclerosis, as well as other neurological and psychiatric disorders.

In various embodiments, the procedure is performed in conjunction with imaging methods to identify healthy and pathological lymph nodes and lymphatic vessels of the CSF/ISF draining lymphatic system, identify meningeal lymphatic, and target jugular foramen and apertures in the cribriform plate. The preferred imaging methods are the high frequency ultrasound (20 MHz-70 MHz) and contrast MRI.

### EXAMPLES

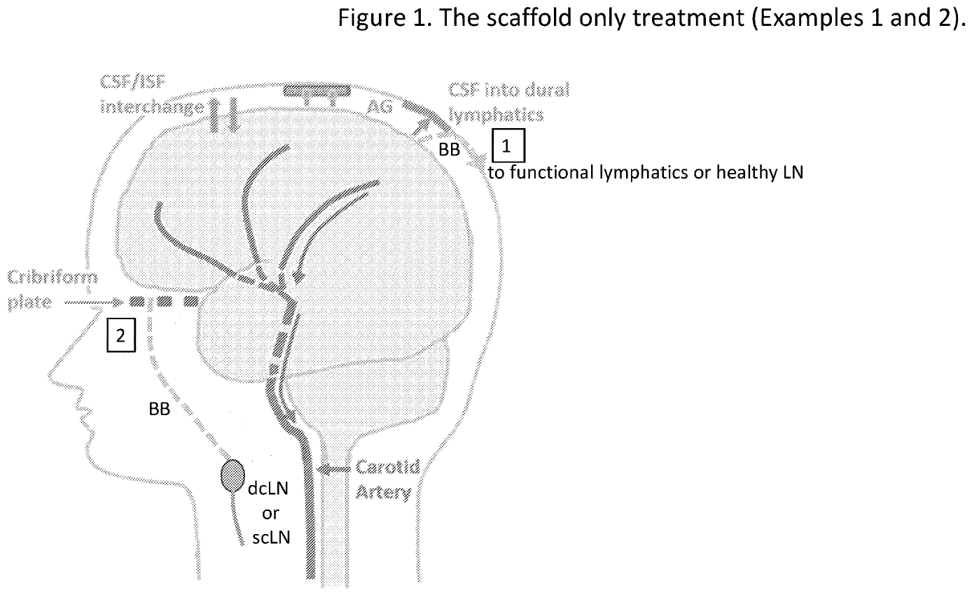

A patient with neurological or psychiatric disorder (for example, AD) is examined by high frequency ultrasound system (20-70 MHz) and/or other diagnostic system (for example, MRI) [references 6,7] that can identify problems with lymphatic drainage of CSF and, specifically, determine the healthy and diseased lymph nodes and lymph vessels in head and neck. Depending on the CSF drainage problems the patient can be treated by implanting biodegradable scaffold supporting directional lymphatic regeneration as depicted in FIGS. 1-6.

### Example 1

Thread-like scaffold is implanted under the skin of the subject head such that the first end of the thread-like scaffold is directed to the functional lymphatics of the “head and neck lymphatic system” and the second end of the thread-like scaffold goes through the skull into the dura mater in the proximity of the meningeal lymphatics, see FIG. 1. For example, the scaffold is made from aligned nanofibrillar biopolymer comprising aligned nanofibrillar collagen type I.

The scaffold can also comprise a growth factor (for example, VEGFC) or a nucleic acid (for example, HGF-mRNA) or cells promoting lymphangiogenesis (for example, endothelial or endothelial progenitor cells) as demonstrated in [references 8-15]. The gradient of VEGFC growth factor will further enhance directional regeneration. The sustain release of HGF modified messenger RNA (HGF-mRNA) and delivered endothelial cells will speed up lymphatic regeneration and form a complementary lymphatic pathway through the scull before the bone regeneration.

### Example 2

The thread-like scaffold is implanted such that the first end of the thread-like scaffold is inserted into cribriform plate or into jugular foramen and the second end of the thread-like scaffold goes into a functional cervical lymph node, see FIG. 1. The ends of the scaffold are sutured to the soft tissue. The guided delivery of the scaffold is conducted under the guidance of the high frequency ultrasound.

### Example 3

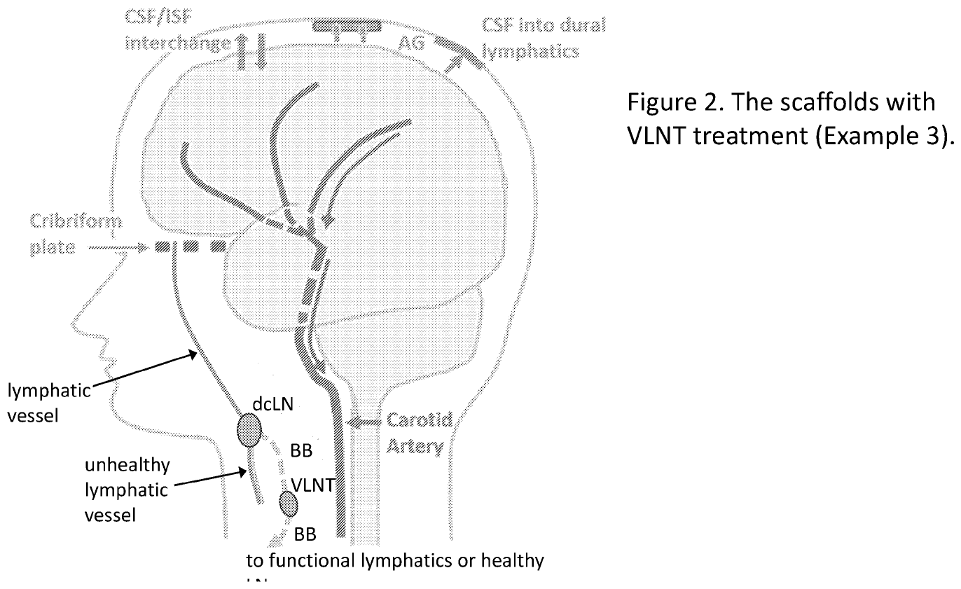

The scaffolds are implanted to augment the transferred lymph node and improve its integration into lymphatic system, see FIG. 2. In this example first scaffold is bridging the transferred lymph node with a deep cervical lymph node which has a viable afferent and unhealthy efferent lymphatic channel, and second scaffold is connected transferred lymph node with a healthy lymphatics to induce a formation of new efferent lymphatic vessel. The healthy autologous lymph node can be transferred from groin or some another suitable donor place. A supraclavicular lymph node flap can be used as a propeller lymphatic tissue flap [reference 16]. A supraclavicular lymph node flap is frequently used for lymphedema treatment without complications in the donor site. The scaffold connection between two lymph nodes in cervical area has been tested in mouse model and formation of new lymphatic channels between deep cervical lymph node and superficial cervical lymph node has been demonstrated in two months after the implantation of aligned nanofibrillar collagen scaffold (BioBridge™).

### Example 4

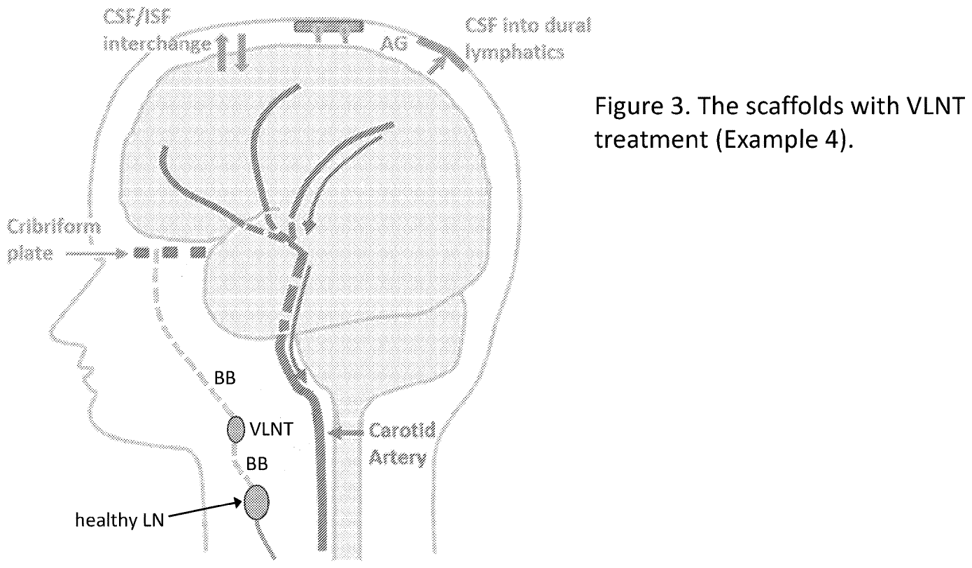

The scaffolds implanted to augment the transferred lymph node and improve its integration into lymphatic system, see FIG. 3. The first scaffold is implanted to connect the lymphatics of cribriform plate with the transferred lymph node and the second scaffold is implanted to connect transferred lymph node with a healthy lymphatics below transferred lymph node to induce a formation of new efferent lymphatic vessel.

### Example 5

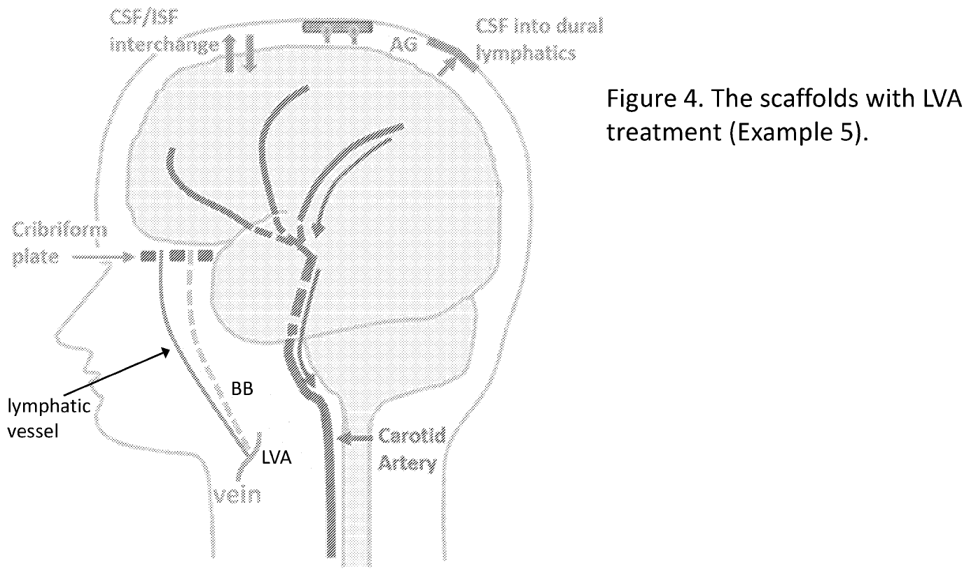

The implanted thread-like scaffold and LVA in the neck region connecting a healthy lymphatic vessel going from meningeal lymphatics with a vein are presented in the FIG. 4. The scaffold is implanted to enhance the flow of lymph fluid from meningeal lymphatic to the LVA area. There is clinical evidence [17,18] that in patients with upper and lower limb lymphedema the implantation of aligned nanofibrillar collagen scaffold (BioBridge™) in the vicinity of LVA induces a formation of new lymphatic vessels.

### Example 6

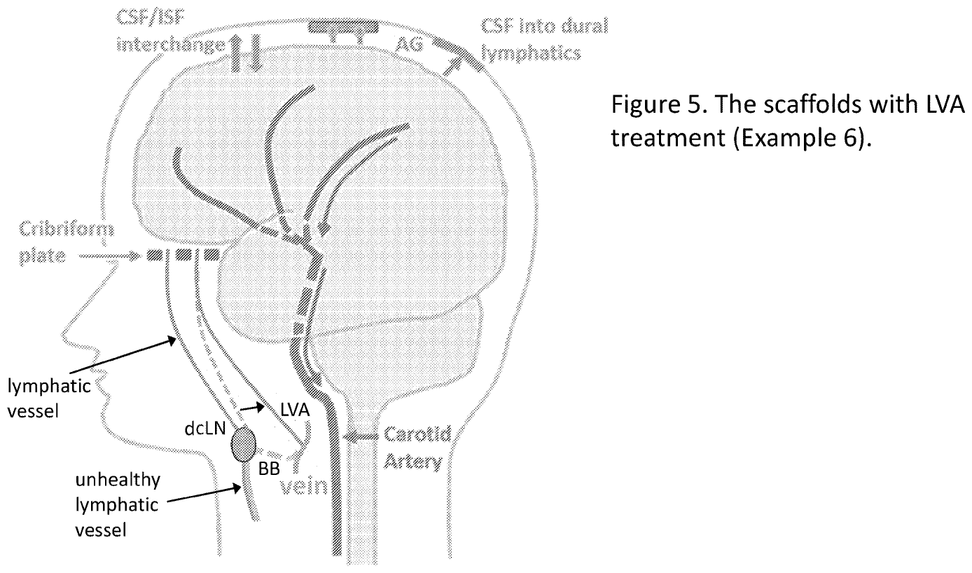

The implanted thread-like scaffold and LVA in the neck region connecting a healthy lymphatic vessel going from meningeal lymphatics with a vein are presented in the FIG. 5. The scaffold is implanted to connect a deep cervical lymph node having a degraded efferent lymphatic vessel to the LVA area inducing a formation of a new efferent lymphatic vessel and enhancing CNS antigen surveillance.

### Example 7

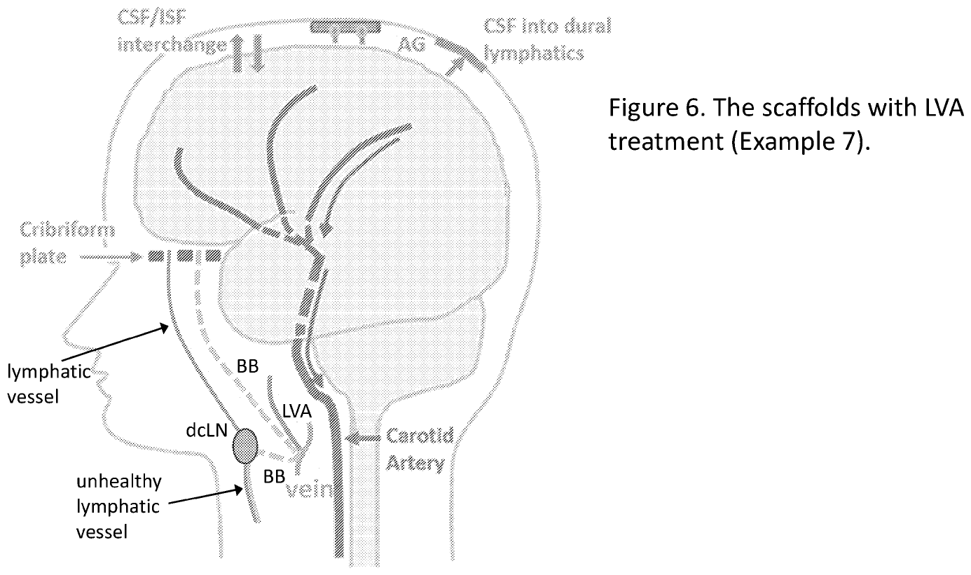

The implanted thread-like scaffolds and LVA in the neck region connecting a healthy lymphatic vessel with a vein are presented in the FIG. 6. The first scaffold is implanted to connect a deep cervical lymph node having a degraded efferent lymphatic vessel to the LVA area inducing a formation of a new efferent lymphatic vessel and enhancing CNS antigen surveillance. The second scaffold is implanted to connect meningeal lymphatics with a vicinity of LVA inducing a formation of a new lymphatic vessel going from meningeal lymphatics to a vicinity of LVA and enhancing the flow of CSF.

### Example 8

Human recombinant VEGF-C was reconstituted in 4 mM HCl and mixed with purified porcine collagen type I solution (3 mg/ml in 4 mM HCl). Collagen microspheres (CMS) filled with VEGF-C (20 ng VEGF-C per mg collagen) were prepared by Spray Dryer System with the microparticle size 1-2 microns. The material was tested in vitro for VEGF-C release by ELISA and for VEGF-C bioactivity in EC culture. The ratio of VEGF-C to collagen was selected based on the dose delivered in vivo in mice AD model [references 19]. The suspension of CMS in PBS solution with collagen concentration 40 mg/ml was injectable by 30G needle into a dura mater forming an injectable scaffold which provides a sustain release of VEGF-C to increase the meningeal lymphatics capacity and flow in CNS.

It will be appreciated that the term “present invention” or “invention” as used herein should not be construed to mean that only a single invention having a single essential element or group of elements is presented. Similarly, it will also be appreciated that the term “present invention” or “invention” encompasses a number of separate innovations, which can each be considered separate inventions. Although the present invention has been described in detail with regards to the preferred embodiments and drawings thereof, it should be apparent to those skilled in the art that various adaptations and modifications of embodiments of the present invention may be accomplished without departing from the spirit and the scope of the invention.

Accordingly, it is to be understood that the detailed description and the accompanying drawings as set forth hereinabove are not intended to limit the breadth of the present invention, which should be inferred only from the following claims and their appropriately construed legal equivalents.

### REFERENCES

1. Baranello R, Bharani K, Padmaraju V, et al. Amyloid-beta protein clearance and degradation (ABCD) pathways and their role in Alzheimer's disease. Curr Alzheimer Res 2015; 12(1): 32-46. http://dx.doi.org/10.2174/1567205012666141218140953 PMID: 25523424.
2. Chachaj A, Ga̧siorowski K, Szuba A, Sieradzki A, Leszek J. Lymphatic system in the brain clearance mechanisms—new therapeutic perspectives for Alzheimer's disease. Curr Neuropharmacol 2022; http://dx.doi.org/10.2174/1570159X20666220411091332 PMID: 35410605.
3. Maloveská M, Humeník F, Vikartovská Z, et al. Brain fluid channels for metabolite removal. Physiol Res 2022; 71(2): 199-208. http://dx.doi.org/10.33549/physiolres.934802 PMID: 35344669.
4. Pappolla M, Sambamurti K, Vidal R, Pacheco-Quinto J, Poeggeler B, Matsubara E. Evidence for lymphatic Aβ clearance in Alzheimer's transgenic mice. Neurobiol Dis 2014; 71: 215-9. http://dx.doi.org/10.1016/j.nbd.2014.07.012 PMID: 25102344.
5. Pappolla M A, Matsubara E, Vidal R, et al. Melatonin treatment enhances Aβ lymphatic clearance in a transgenic mouse model of amyloidosis. Curr Alzheimer Res 2018; 15(7): 637-42. http://dx.doi.org/10.2174/1567205015666180411092551 PMID: 29637859.
6. Bianchi A, Visconti G, Hayashi A, Santoro A, Longo V, Salgarello M. Ultra-High frequency ultrasound imaging of lymphatic channels correlates with their histological features: A step forward in lymphatic surgery. J Plast Reconstr Aesthet Surg. 2020 Sep.; 73(9): 1622-1629.
7. Carare R O, Aldea R, Agarwal N, Bacskai B J, Bechman I, Boche D, Bu G, Bulters D, Clemens A. Clearance of interstitial fluid (ISF) and CSF (CLIC) group-part of Vascular Professional Interest Area (PIA): Cerebrovascular disease and the failure of elimination of Amyloid-β from the brain and retina with age and Alzheimer's disease-Opportunities for Therapy. Alzheimers Dement (Amst). 2020; 12(1): e12053.
8. Hadamitzky C, Zaitseva T S, Bazalova-Carter M, Paukshto M V, Hou L, Strassberg Z, Ferguson J, Matsuura Y, Dash R, Yang P C, Kretchetov S, Vogt P M, Rockson S G, Cooke J P, Huang N F. Aligned nanofibrillar collagen scaffolds—Guiding lymphangiogenesis for treatment of acquired lymphedema. Biomaterials 2016, 102, 259-267.
9. Christiana Raymond-Pope, Tatiana Zaitseva, Eduard Yakubov, Daniel Hoffman, Benjamin Corona, Michael Paukshto, Sarah Greising. Optimizing growth factor mRNA delivery in a bioengineered aligned scaffold to treat volumetric muscle loss. 2022 Military Health System Research Symposium. Poster. 2022. https://news.cehd.umn.edu/raymond-pope-presents-at-the-2022-military-health-system-research-symposium/
10. Zaitseva T S, Yang G, Dionyssiou D, Zamani M, Sawamura S, Yakubov E, Ferguson J, Hallett R L, Fleischmann D, Paukshto M V, Huang N F. Delivery of hepatocyte growth factor mRNA from nanofibrillar scaffolds in a pig model of peripheral arterial disease. Regen Med. 2020 06; 15(6): 1761-1773.
11. Hu C, Zaitseva T S, Alcazar C, Tabada P, Sawamura S, Yang G, Borrelli M R, Wan D C, Nguyen D H, Paukshto M V, Huang N F. Delivery of Human Stromal Vascular Fraction Cells on Nanofibrillar Scaffolds for Treatment of Peripheral Arterial Disease. Front Bioeng Biotechnol. 2020; 8: 689.
12. Zaitseva T S, Alcazar C, Zamani M, Hou L, Sawamura S, Yakubov E, Hopkins M, Woo Y J, Paukshto M V, Huang N F. Aligned Nanofibrillar Scaffolds for Controlled Delivery of Modified mRNA. Tissue Eng Part A. 2019 01; 25(1-2): 121-130.
13. Ngan F. Huang, Tatiana Zaitseva, Cynthia Alcazar, Maedeh Zamani, Eduard Yakubov, Greg King, Michael Paukshto. Delivery of Modified mRNA-Loaded Aligned Nanofibrillar Scaffolds for Treatment of Traumatic Muscle Injury. Society for Biomaterials 2018 Annual Meeting. Poster.
14. Nakayama K H, Hong G, Lee J C, Patel J, Edwards B, Zaitseva T S, Paukshto M V, Dai H, Cooke J P, Woo Y J, Huang N F. Aligned-Braided Nanofibrillar Scaffold with Endothelial Cells Enhances Arteriogenesis. ACS Nano. 2015 Jul. 28; 9(7): 6900-8.
15. Huang N F, Okogbaa J, Lee J C, Jha A, Zaitseva T S, Paukshto M V, Sun J S, Punjya N, Fuller G G, Cooke J P. The modulation of endothelial cell morphology, function, and survival using anisotropic nanofibrillar collagen scaffolds. Biomaterials. 2013. 34: 4038-4047.
16. Dimitrios Dionyssiou, Dung Nguyen, Anastasios Topalis, Peter Deptula, Michael Paukshto, Tatiana Zaitseva, Efterpi Demiri, Aggeliki Cheva, Stanley Rockson. Treating rat lymphedema with a propeller lymphatic tissue flap combined with nanofibrillar collagen scaffolds. J Reconstr Microsurg. 2023 Jul. 5.
17. Nguyen D, Zaitseva T S, Zhou A, Rochlin D, Sue G, Deptula P, Tabada P, Wan D, Loening A, Paukshto M, Dionyssiou D. Lymphatic regeneration after implantation of aligned nanofibrillar collagen scaffolds: Preliminary preclinical and clinical results. J Surg Oncol. 2022 Feb.; 125(2): 113-122.
18. Nguyen D H, Zhou A, Posternak V, Rochlin D H. Nanofibrillar Collagen Scaffold Enhances Edema Reduction and Formation of New Lymphatic Collectors after Lymphedema Surgery. Plast Reconstr Surg. 2021 Dec. 1; 148(6): 1382-1393.
19. Da Mesquita S, Louveau A, Vaccari A, Smirnov I, Cornelison R C, Kingsmore K M, Contarino C, Onengut-Gumuscu S, Farber E, Raper D, Viar K E, Powell R D, Baker W, Dabhi N, Bai R, Cao R, Hu S, Rich S S, Munson J M, Lopes M B, Overall C C, Acton S T, Kipnis J. Functional aspects of meningeal lymphatics in ageing and Alzheimer's disease. Nature. 2018 08; 560(7717): 185-191.
20. Bianchi L M G, Irmici G, CèM, D'Ascoli E, Della Pepa G, Di Vita F, Casati O, Soresina M, Menozzi A, Khenkina N, Cellina M. Diagnosis and Treatment of Post-Prostatectomy Lymphedema: What's New? Curr Oncol. 2023 Apr. 25; 30(5): 4512-4526.
21. Inchauste S M, Nguyen D H, and Ming-Huei Cheng. Animal Study and Cadaver Dissection of Lymphedema. In book: Principle of Lymphatic System. Elsevier, 2022.
22. Chin-Yu Yang, Ines E. Tinhofer, Dung Nguyen, Ming-Huei Cheng. Enhancing lymphangiogenesis and lymphatic drainage to vascularized lymph nodes with nanofibrillar collagen scaffolds. J Surg Oncol. 2022; 1-7.
23. Deptula P, Zhou A, Posternak V, He H, Nguyen D. Multimodality Approach to Lymphedema Surgery Achieves and Maintains Normal Limb Volumes: A Treatment Algorithm to Optimize Outcomes. J Clin Med. 2022 Jan. 25; 11(3): 598.
24. Attier-Zmudka J, Sérot J M, Valluy J, Saffarini M, Macaret A S, Diouf M, Dao S, Douadi Y, Malinowski K P, Balédent O. Decreased Cerebrospinal Fluid Flow Is Associated With Cognitive Deficit in Elderly Patients. Front Aging Neurosci. 2019; 11: 87.
25. Loureiro-Campos E, Pinto L, Mendanha Falcão A. CSF circulation regulates depression: do not disturb the flow! Mol Psychiatry. 2021 12; 26(12): 7072-7073.
26. Noé F M, Marchi N. Central nervous system lymphatic unit, immunity, and epilepsy: Is there a link? Epilepsia Open. 2019 Mar.; 4(1): 30-39.
27. Kelly L, Brown C, Michalik D, Hawkes CA, Aldea R, Agarwal N, Salib R, Alzetani A, Ethell D W, Counts S E, de Leon M, Fossati S, Koronyo-Hamaoui M, Piazza F, Rich S A, Wolters F J, Snyder H, Ismail O, Elahi F, Proulx S T, Verma A, Wunderlich H, Haack M, Dodart J C, Mazer N, Carare R O. Clearance of interstitial fluid (ISF) and CSF (CLIC) group-part of Vascular Professional Interest Area (PIA), updates in 2022-2023. Cerebrovascular disease and the failure of elimination of Amyloid-β from the brain and retina with age and Alzheimer's disease: Opportunities for therapy. Alzheimers Dement. 2023 October 28.

1. A method of increasing flow of fluid in the central nervous system of a subject in need thereof, the method comprising:
implanting a biocompatible scaffold into the subject head or neck wherein the scaffold induces a directional lymphatic regeneration and formation of a complementary lymphatic pathway,
thereby resulting in an additional flow of cerebrospinal and/or extracellular fluid from the central nervous system into the circulatory system of the subject.

2. A method of increasing turnover of cerebrospinal fluid of a subject in need thereof, the method comprising:
implanting a biocompatible scaffold into the subject head or neck wherein the scaffold induces a directional lymphangiogenesis,
thereby resulting in an increase in turnover of the subject cerebrospinal fluid.

3. The method of claim 1 or claim 2, wherein said subject has Alzheimer's Disease or Parkinson's disease or stroke or post-traumatic brain injury or some other relevant neurological disorder affected by the brain clearance system.

4. A method of claim 1, wherein the scaffold supports a flow of cerebrospinal and/or extracellular fluid of the subject and induces a formation of a complementary lymphatic pathway from the dura mater to the head and neck circulatory system of the subject, thereby increasing fluid flow in the central nervous system of the subject.

5. A method of claim 1 or claim 2, wherein the scaffold is comprised of a thread-like biodegradable scaffold or a set of the thread-like biodegradable scaffolds.

6. A method of claim 1 or claim 2, wherein the scaffold is an injectable scaffold.

7. A method of claim 5, wherein the thread-like scaffold comprises a bundle of threads, or multi-filaments, or fibers, or fibrils made from biodegradable materials such that the bundle supports a capillary flow of cerebrospinal and/or extracellular fluid along the bundle, in the bundle direction.

8. A method of claim 5, wherein the thread-like scaffold has a multi-lumen or multi-channel structure such that the thread-like scaffold enabling a flow of cerebrospinal and/or extracellular fluid in the thread-like scaffold direction.

9. A method of claim 5, wherein the thread-like scaffold comprising biodegradable materials selected from the group consisting of collagen, fibronectin, fibrin, laminin, elastin, hyaluronic acid, chitosan, silk, peptides, block copolymers, lactide and glycolide polymers, caprolactone polymers, hydroxybutyric acids, polyanhydrides and polyesters, polyphosphates, porous silicon, polyphosphoesters, poly(ethylene glycol) (PEG) and poly(ethylene oxide) (PEO) including PEG and PEO with different end functionalities, as well as bifunctional cross-linkers and crosslinking agents, or the combinations of the above materials.

10. A method of claim 5, wherein the thread-like scaffold comprises aligned nanofibrillar biopolymer.

11. A method of claim 1 or claim 2, wherein the scaffold comprises a growth factor promoting lymphangiogenesis.

12. A method of claim 1 or claim 2, wherein the scaffold comprises a nucleic acid promoting lymphangiogenesis.

13. A method of claim 1 or claim 2, wherein the scaffold comprises cells that repair lymphatics.

14. A method of claim 13, wherein the cells are autologous or allogeneic endothelial or endothelial progenitor cells.

15. A method of claim 2, wherein the scaffold is implanted into the dura mater to increase the capacity of meningeal lymphatics and enhance the turnover of cerebrospinal fluid.

16. A method of claim 4, wherein the thread-like scaffold is implanted under the skin of the subject head such that the first end of the thread-like scaffold is kept in the proximity of the functional lymphatics of the head and neck lymphatic system and the second end of the thread-like scaffold goes through the skull into the dura mater in the proximity of the meningeal lymphatics.

17. A method of claim 6, wherein the injectable scaffold comprising biodegradable materials selected from the group consisting of collagen, fibronectin, fibrin, laminin, elastin, hyaluronic acid, chitosan, silk, peptides, block copolymers, lactide and glycolide polymers, caprolactone polymers, hydroxybutyric acids, polyanhydrides and polyesters, polyphosphates, porous silicon, polyphosphoesters, poly(ethylene glycol) (PEG) and poly(ethylene oxide) (PEO) including PEG and PEO with different end functionalities, as well as bifunctional cross-linkers and crosslinking agents, or the combinations of the above materials.

18. A method of claim 6, wherein the injectable scaffold comprising collagen microspheres with a growth factor or a nucleic acid or cells promoting lymphangiogenesis.

19. A method of claim 11, wherein the growth factor is the human VEGFC/D, or fibroblast growth factor, or hepatocyte growth factor, or platelet-derived growth factor, or insulin-like growth factor, or other lymphangiogenesis promoting growth factor.

20. A method of claim 1, wherein the increasing flow of fluid in the central nervous system of a subject is combined with immunotherapy or other methods removing waste from central nervous system and protecting from infection.

21. A method of claim 2, wherein the increasing turnover of cerebrospinal fluid in the central nervous system of a subject is combined with immunotherapy or other methods removing waste from central nervous system and protecting from infection.

22. A method of claim 1 or claim 2, wherein the implantation of biocompatible scaffold is performed in conjunction with imaging methods to identify healthy and pathological lymph nodes and lymphatic vessels of the CSF/ISF draining lymphatic system and/or to identify meningeal lymphatic and/or to identify and target jugular foramen and apertures in the cribriform plate.

23. A method of claim 18, wherein the growth factor is the human VEGFC/D, or fibroblast growth factor, or hepatocyte growth factor, or platelet-derived growth factor, or insulin-like growth factor, or other lymphangiogenesis promoting growth factor.
